(19) United States
(12) Patent Application Publication
(10) Pub. No.: US 2019/0208984 A1
(43) Pub. Date: Jul. 11, 2019

(54) ENDOSCOPE

(71) Applicant: OLYMPUS CORPORATION, Tokyo (JP)

(72) Inventors: Mamoru MACHIYA, Tokyo (JP); Kyota SAWAI, Tokyo (JP)

(73) Assignee: OLYMPUS CORPORATION, Tokyo (JP)

(21) Appl. No.: 16/355,968

(22) Filed: Mar. 18, 2019

Related U.S. Application Data
(63) Continuation of application No. PCT/JP2017/036343, filed on Oct. 5, 2017.

(30) Foreign Application Priority Data
Oct. 11, 2016 (JP) 2016-200263

(51) Int. Cl. A61B 1/008; A61B 1/005; G02B 23/24; A61B 1/00

(52) U.S. Cl. CPC A61B 1/008; A61B 1/0052; A61B 1/00066; A61B 1/00147; G02B 23/2476

(57) ABSTRACT

An endoscope of the present invention includes: a cylindrical member disposed at a distal end of a bending portion of an insertion portion; a wire inserted into the cylindrical member; a protruding portion provided by cutting and bending a predetermined part of an outer peripheral surface of the cylindrical member in a direction of a central axis; an outer peripheral surface-side concave where a distal end portion of the wire is disposed; and a guide groove including an end part face provided at a predetermined position of the outer peripheral surface-side concave, the guide groove being elongated along the central axis and larger in width than a diameter of the wire, the guide groove being configured to guide the wire to the outer peripheral surface-side concave.

### CROSS REFERENCE TO RELATED APPLICATION

This application is a continuation application of PCT/JP2017/036343 filed on Oct. 5, 2017 and claims benefit of Japanese Application No. 2016-200263 filed in Japan on Oct. 11, 2016, the entire contents of which are incorporated herein by this reference.

### BACKGROUND OF THE INVENTION

### 1. Field of the Invention

The present invention relates to an endoscope in which a wire distal end portion for bending a bending portion is fixed to a distal end piece.

### 2. Description of the Related Art

An endoscope has an elongated insertion portion. A distal end portion on a distal end side of the insertion portion incorporates components such as an observation optical system for picking up an observed image of an observation site. The insertion portion may have a bending portion provided on a proximal end side of the distal end portion. In such an endoscope which has the insertion portion provided with the bending portion, it is possible to easily insert the insertion portion into deep regions of an observation site when the bending portion is appropriately bent, and to turn the observation optical system provided at the distal end portion in a desired direction.

The bending portion is formed from, for example, a bending piece group including a plurality of bending pieces which are connected with each other in a turnable manner, and a bending rubber which covers the bending piece group. The bending piece group bends, for example, in two directions including an upward direction and a downward direction, or in four directions including an upward direction, a downward direction, a rightward direction, and a leftward direction. The bending portion which bends in two directions allows insertion of two wires, which correspond to the bending directions, into the bending piece group, and the bending portion configured to bend in four directions allows insertion of four wires into the bending piece group.

Wire distal end portions of the plurality of wires are fixed to wire fixing portions of a distal end piece which constitutes a most distal end of the bending piece group, while proximal ends of the wires are fixed to a bending operation device arranged inside an operation portion which is provided on a proximal end side of the insertion portion. The bending portion bends, when a user operates the bending operation device so as to draw or loose the wires.

A second embodiment in Japanese Patent Application Laid-Open Publication No. 2001-104238 discloses a bending portion which has a first bending piece at a most distal end. The first bending piece has, for example, two notch sections collinearly provided in an axis direction. The notch sections include hole portions that allow insertion of an angle wire, and the notch sections which receive a distal end of the angle wire are caulked from an inner peripheral side to an outer peripheral side of the bending piece so as to be deformed in such a manner that the notch sections dig at the angle wire. As a result, the angle wire is fixed so as to prevent the distal end portion from dropping off even when the angle wire is pulled. In addition, the amount of radially inward protrusion of the notch sections, which are formed by notching an inner peripheral surface of the first bending piece, becomes equal to or less than the size obtained by adding a diameter of the angle wire and a thickness of the first bending piece, and therefore the amount of protrusion becomes extremely small.

### SUMMARY OF THE INVENTION

In order to accomplish the above object, an endoscope in one aspect of the present invention includes: a cylindrical member disposed at a distal end of a bending portion provided on a distal end side of an insertion portion; a wire inserted into the cylindrical member along a longitudinal axis of the insertion portion; a protruding portion provided in the cylindrical member, the protruding portion being provided by cutting and bending a predetermined part of an outer peripheral surface of the cylindrical member in a direction of a central axis; an outer peripheral surface-side concave provided on the protruding portion so as to allow disposition of a distal end portion of the wire; and a guide groove provided in the protruding portion, the guide groove including an end part face provided at a predetermined position of the outer peripheral surface-side concave, the guide groove being elongated along the central axis and larger in width than a diameter of the wire, the guide groove being configured to guide the wire inserted into the cylindrical member to the outer peripheral surface-side concave.

### BRIEF DESCRIPTION OF THE DRAWINGS

FIG. 1 is a view illustrating a configuration example of an endoscope having a bending portion in an insertion portion;

FIG. 2 is a longitudinal cross-sectional view illustrating the configuration of a distal end side of the insertion portion;

FIG. 3 is a cross-sectional view along [3]-[3] line of FIG. 2; FIG. 4 is a view illustrating a protruding portion, which is provided in a distal end piece by cutting and bending in the direction of a central axis, viewed in the direction of arrow [4] of FIG. 2;

FIG. 5 is a view of the distal end piece viewed from a proximal end side as shown by arrow [5] of FIG. 4;

FIG. 6 is a cross-sectional view along arrow line [6]-[6] of FIG. 4;

FIG. 7 is a view illustrating the state where a wire distal end portion is guided to a cut-and-bent space, and the state where the wire distal end portion is placed and fixed on a placement portion of an outer peripheral surface-side concave;

FIG. 8 is a cross-sectional view of FIG. 7 showing a bonding portion which fixes the wire distal end portion to the outer peripheral surface-side concave; and

FIG. 9 is a view illustrating a thin wall portion of the protruding portion.

### DETAILED DESCRIPTION OF THE PREFERRED EMBODIMENT

An embodiment of the present invention will be described hereinbelow with reference to the drawings.

Note that in the respective drawings used for the following description, different scales may be used for each component member in order to present each component member in a size recognizable on the drawings. In other words, the present invention is not limited only to the number of the component members, shapes of the component members, size ratios of the component members, and relative positional relationship among the component members described in the drawings.

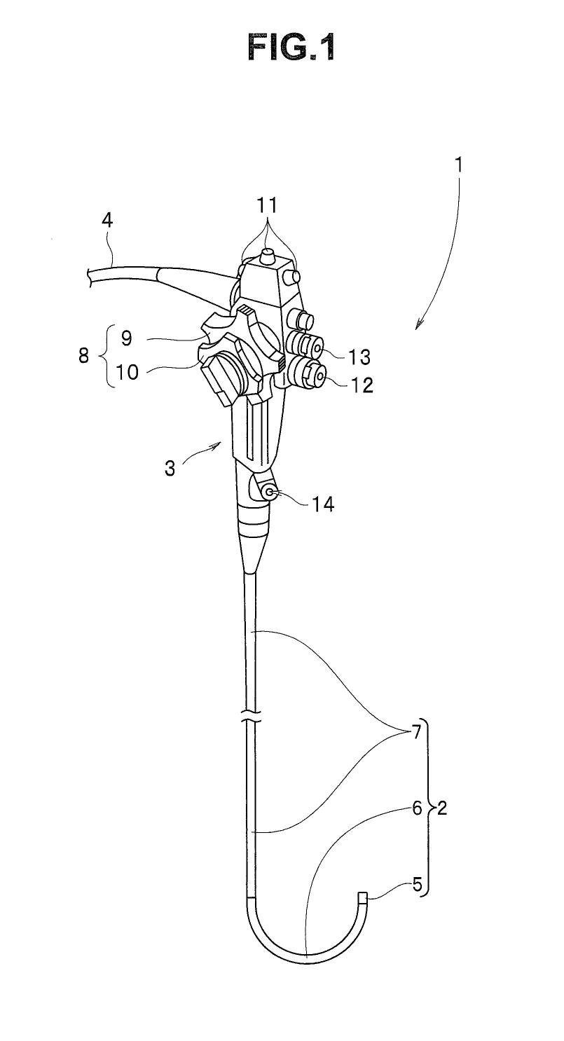

As illustrated in FIG. 1, an endoscope 1 has an insertion portion 2, an operation portion 3, and a universal cable 4. The insertion portion 2 is an elongated long member inserted to a site to be observed. In the insertion portion 2, a distal end portion 5, a bending portion 6, and a flexible tube portion 7 are connected in order from the distal end side.

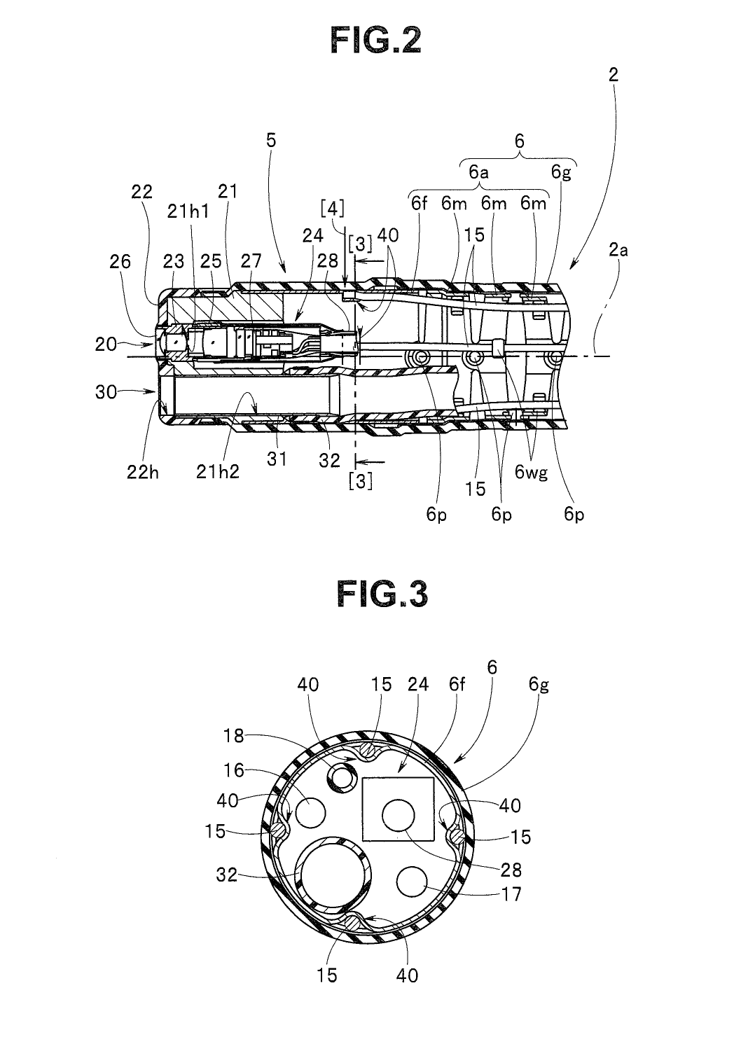

The distal end portion 5 incorporates an illumination optical system (not illustrated) including a light guide, and an observation optical system (see reference numeral 20 of FIG. 2) including an image pickup apparatus. The bending portion 6 is configured to be bendable in four directions including an upward direction, a downward direction, a rightward direction, and a leftward direction. The flexible tube portion 7 is a tubular member which is long and flexible.

The operation portion 3 is provided with a bending operation device 8, various switches 11, an air/water feeding button 12, a suction button 13, and the like. The bending operation device 8 is configured to include an upward/downward bending knob 9 and a rightward/leftward bending knob 10. Examples of the various switches 11 include a release switch, a freeze switch, and an observation mode selector switch for switching normal observation and fluorescence observation. Reference numeral 14 designates a treatment instrument insertion opening.

Note that the bending portion 6 may be configured to be bendable in two directions including an upward direction and a downward direction. In this case, the bending operation device 8 includes the upward/downward bending knob 9 or a bending lever (not illustrated), for example.

The universal cable 4 extends from a lateral portion of the operation portion 3. At an end portion of the universal cable 4, an unillustrated endoscope connector is provided.

As shown in FIG. 2, the distal end portion 5 includes a distal end component member 21 that is a rigid member. A distal end cover 22 which is a non-conductive member made of, for example, resin is firmly installed on the distal end side of the distal end component member 21. Reference numeral 22h designates a cover throughhole, which constitutes a treatment instrument insertion channel 30.

The distal end component member 21 is made of metal such as stainless steel, made of hard resin, or made of ceramics. In the distal end component member 21, throughholes parallel to a longitudinal axis 2a of the insertion portion 2, such as an observation optical system throughhole 21h1, an illumination optical system throughhole (not illustrated), an air/water feeding throughhole (not illustrated), and a treatment instrument insertion throughhole 21h2, are formed at predetermined positions.

The observation optical system throughhole 21h1 is, for example, a stepped hole including a small diameter hole and a large diameter hole in order from the distal end side. An objective lens frame 23 of the observation optical system 20 is firmly installed in the small diameter hole, and an image pickup apparatus 24 of the observation optical system 20 is disposed in the large diameter hole. Reference numeral 25 designates an image pickup frame, reference numeral 26 designates an observation lens, and reference numeral 27 designates an image pickup device. In the present embodiment, the image pickup device 27 is, for example, a CCD. Reference numeral 28 designates a signal cable.

A connecting tube 31 which constitutes the treatment instrument insertion channel 30 is firmly installed in the treatment instrument insertion throughhole 21h2. The proximal end side of the connecting tube 31 protrudes from a proximal end surface of the distal end component member 21. A distal end portion of a treatment instrument channel tube 32 which constitutes the treatment instrument insertion channel 30 is fixed to a protruding proximal end portion of the connecting tube 31. The treatment instrument channel 30 allows insertion of treatment instruments, such as a reusable puncture needle and grasping forceps.

A distal end piece 6f which constitutes the bending portion 6 is firmly installed on the proximal end side of the distal end component member 21. The bending portion 6 mainly includes a bending piece group 6a and a bending rubber 6g. The bending piece group 6a includes cylindrical members including the distal end piece 6f, a plurality of middle pieces 6m, and a proximal end piece (not illustrated) in order from the distal end side. The respective pieces which constitute the bending piece group 6a are turnably and pivotably supported by connecting pins 6P. The bending rubber 6g covers the outer peripheral surface side of the bending piece group 6a.

Reference numeral 15 designates wires. The wires 15 are set to have a predetermined diameter. Four wires 15 are provided corresponding to upward, downward, rightward, and leftward bending directions. Along the longitudinal axis 2a of the insertion portion 2, the four wires 15 extend through the distal end piece 6f, the middle pieces 6m, the proximal end piece (not illustrated), and the flexible tube portion 7 into the operation portion 3 provided on the proximal end side of the flexible tube portion 7.

Wire distal end portions 15a that are distal end portions of the four wires 15 are fixed to protruding portions 40 that are wire distal end portion fixing portions provided in the distal end piece 6f. The protruding portions 40 are provided by cutting and bending an outer peripheral surface 6fo, which is a predetermined part of the distal end piece 6f, in the direction of a central axis 6fa of the distal end piece 6f from the side of the outer peripheral surface 6fo.

The central axis 6fa is aligned with the longitudinal axis 2a of the insertion portion 2 in FIG. 2.

As shown in FIG. 3, the protruding portions 40 are provided at predetermined positions, which correspond to the upward, downward, rightward, and leftward directions, around the distal end piece 6f. The distal end piece 6f allows insertion of endoscope internal components, including not only the image pickup apparatus 24 and the treatment instrument channel tube 32 but also, for example, two light guide fiber bundles 16 and 17, and an air/water feeding tube 18, along the longitudinal axis 2a of the insertion portion 2.

Note that reference numeral 6wg of FIG. 2 designates a wire guide, which is provided in the middle pieces 6m.

Here, the distal end piece 6f is described with reference to FIGS. 4 to 6.

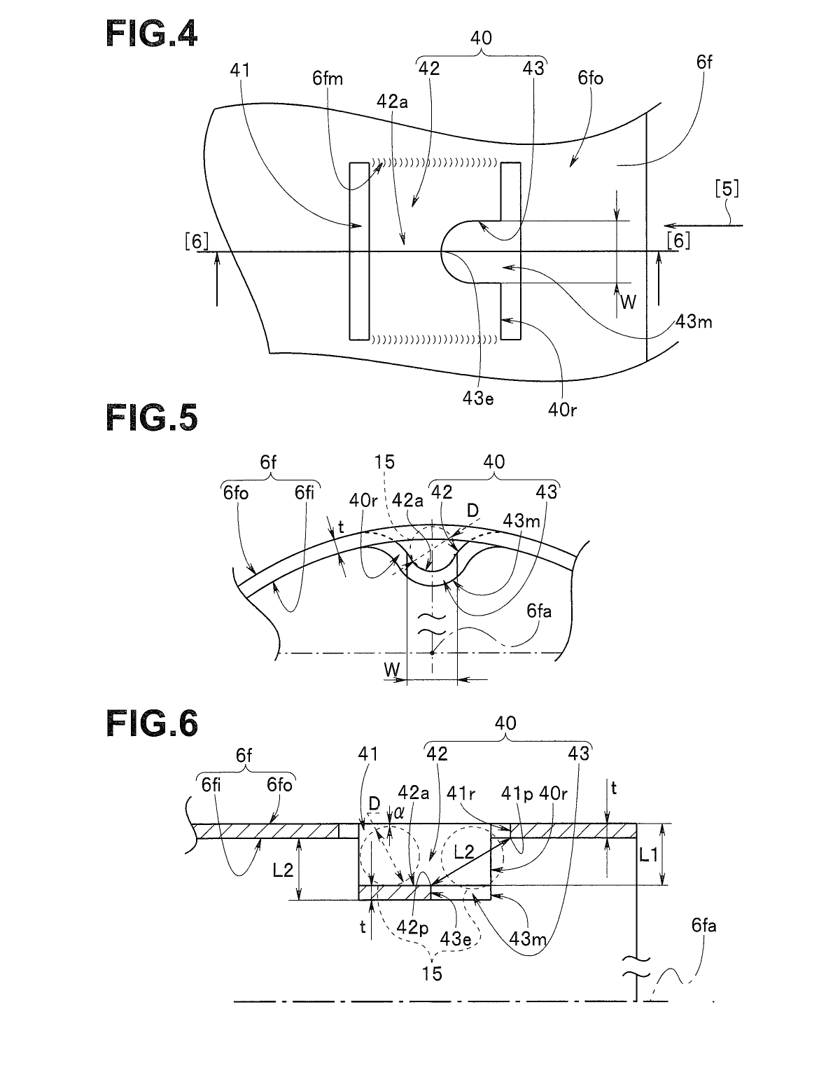

As shown in FIG. 4, one typical protruding portion 40 is provided in a predetermined part of the outer peripheral surface 6fo of the distal end piece 6f. As shown in FIGS. 5 and 6, the protruding portion 40 is provided by cutting and bending a predetermined portion of the outer peripheral surface 6fo of the distal end piece 6f in the direction of the central axis 6fa from an inner peripheral surface 6fi of the distal end piece 6f, so that the protruding portion 40 has a predetermined amount of protrusion in a predetermined shape.

As shown in FIGS. 4 and 6, since the protruding portion 40 is provided in the distal end piece 6f, the distal end piece 6f is provided with a cut-and-bent space 41 having a distal end piece outer peripheral-side opening 6fm. The cut-and-bent space 41 has a function as a wire housing portion.

Accordingly, as shown in FIGS. 4 to 6, the above-stated protruding portion 40 has an outer peripheral surface-side concave 42 and a groove 43. As shown in FIG. 5, the outer peripheral surface-side concave 42 has a placement portion 42a which allows disposition of the distal end portion 15a of one of the wires 15. The placement portion 42a is a curved surface that allows placement of the wire 15 having a diameter D shown with a broken line. The placement portion 42a is provided at the center of the outer peripheral surface-side concave 42, and a curved surface diameter of the placement portion 42a is set to be larger than the diameter D of the wire 15.

As shown in FIG. 6, a distance L1 from a bottom surface of the outer peripheral surface-side concave 42 to the outer peripheral surface 6fo of the distal end piece 6f is set to be larger by a preset clearance a than the diameter D of the wire 15. As a result, in the state where the distal end portion 15a of the wire 15 is disposed on the placement portion 42a in the cut-and-bent space 41, the distal end portion 15a of the wire 15 is prevented from protruding outward from the outer peripheral surface 6fo of the distal end piece 6f.

The amount of protrusion L2 of the protruding portion 40 is a distance from the inner peripheral surface 6fi of the distal end piece 6f to a central side convex 44, which is opposite to the placement portion 42a of the outer peripheral surface-side concave 42. The amount of protrusion L2 can be expressed by L2=(D+α−t)+t=D+α (t is a thickness of the distal end piece 6f). In other words, the amount of protrusion L2 of the protruding portion 40 is substantially equal to the diameter D of the wire 15.

The groove 43 is provided to be elongated along the central axis 6fa. The groove 43 has a groove opening 43m on a proximal end surface side 40r of the protruding portion 40. The groove 43 is a guide groove (the groove 43 is hereinafter also described as the guide groove 43) having a function of guiding the distal end portion 15a of one of the wires 15, which are inserted into the distal end piece 6f, to the cut-and-bent space 41 on the side of the outer peripheral surface-side concave 42.

Therefore, a width W of the guide groove 43 shown in FIG. 5 is set to be larger than the diameter D of the wire 15. In addition, an end part face 43e of the guide groove 43 is set at a predetermined position so that the distal end portion 15a side of the wire 15 is smoothly guided to the outer peripheral surface-side concave 42.

More specifically, the position of the end part face 43e is so set that a distance L3, from an inner peripheral surface end 6fp provided on an opening proximal end-side surface 6fr of the distal end piece outer peripheral-side opening 6fm to an outer peripheral surface-side concave proximal end 42p, is larger than the diameter D of the wire 15. The present embodiment is configured such that the distance L3 is larger than the diameter D of the wire 15 by providing the guide groove 43 in the outer peripheral surface-side concave 42. However, the present embodiment may be configured such that the guide groove is provided not on the side of the outer peripheral surface-side concave 42 but on the side of the inner peripheral surface end 6fp which faces the end part face 43e.

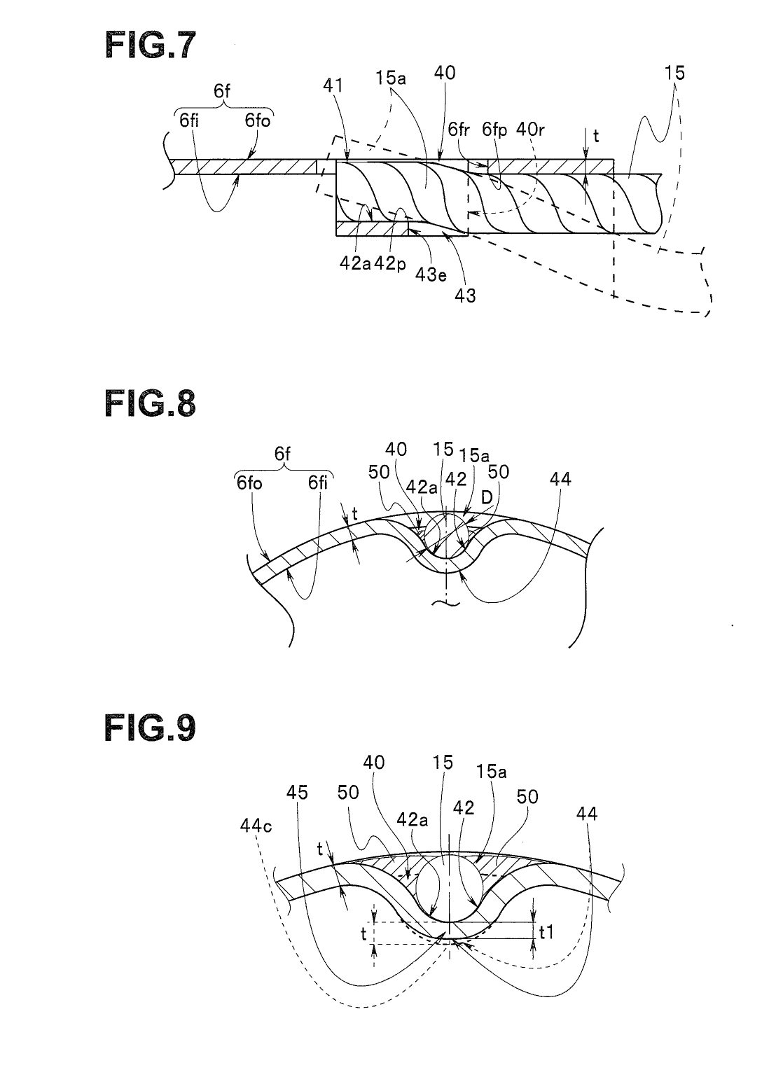

Since the above-stated protruding portion 40 is provided in the distal end piece 6f, it is possible to cause the wire 15 to smoothly pass between the end part face 43e of the guide groove 43 and the proximal end-side surface 6fr of the distal end piece outer peripheral-side opening 6fm, when the distal end portion 15a of the wire 15 is guided from the inner side of the distal end piece 6f into the cut-and-bent space 41 of the distal end piece 6f as shown with a broken line of FIG. 7.

In addition, after the distal end portion 15a of the wire 15 is introduced into the cut-and-bent space 41 of the distal end piece 6f, the distal end portion 15a of the wire 15 can easily be disposed on the placement portion 42a of the outer peripheral surface-side concave 42, without causing the distal end portion 15a to protrude outward from the outer peripheral surface 6fo of the distal end piece 6f as shown with a solid line. At this time, the distal end portion of the wire 15 abuts between the end part face 43e and the proximal end-side surface 6fr, so that the distal end portion can be disposed without causing deformation. This prevents deterioration in durability of the wires 15.

Therefore, it is possible to solder the distal end portion 15a of the wire 15 while retaining the distal end portion 15a in a stable state, so that the distal end portion 15a of the wire 15 can integrally be fixed to the distal end piece 6f with, for example, a silver solder 50 as shown in FIG. 8 in a reliably manner, without causing the distal end portion 15a to protrude outward from the outer peripheral surface 6fo of the distal end piece 6f.

Thus, the distal end piece 6f is provided with the protruding portions 40 each of which includes the outer peripheral surface-side concave 42 and the groove 43, and the distal end portions 15a of the wires 15 are placed on the placement portions 42a of the outer peripheral surface-side concaves 42, respectively. In this state, the distal end portions 15a of the wires 15 are fixed by soldering. Hence, as shown in FIGS. 2 and 3, a part of distal end portion 15a of each of the wires 15 can be disposed in each of the cut-and-bent spaces 41 without causing deformation, so that the amount of protrusion of each of the protruding portions 40 from the inner peripheral surface 6fi can be made substantially equal to the diameter D of each of the wires 15, which is smaller than the value obtained by adding the diameter of each of the wires 15 and the thickness t of the distal end piece 6f. Accordingly, considerable reduction in the amount of protrusion can be achieved.

As a result, reduction in a filling rate of endoscope internal components or reduction in diameter of the bending piece group 6a including the distal end piece 6f can be achieved.

Note that a central portion 44c of the central side convex 44 opposite to the placement portion 42a of the outer peripheral surface-side concave 42 of the protruding portion 40, in which the distal end portion 15a of the wire 15 shown in FIG. 9 is firmly installed, may be plastically deformed in an outer peripheral surface direction to provide the thin wall portion 45. By plastically deforming the vicinity of the placement portion 42a of the protruding portion 40, a thickness t1 in the thin wall portion 45 becomes thinner than the original thickness t. As shown in FIG. 9, the silver solder 50 may also be applied up to just below the inner surface of the outer peripheral surface 6fo in the distal end piece 6f. The silver solder 50 may also be applied to such an extent that the silver solder 50 is piled up higher than the level of the outer peripheral surface 6fo, and then the surface of the silver solder 50 may be ground and polished to be flush with the outer peripheral surface 6fo.

Thus, providing the thin wall portion 45 at the predetermined position in each of the protruding portions 40 can further reduce the amount of protrusion of each of the protruding portions 40 from the inner peripheral surface 6fi by (t−t1), so that further reduction in the filling rate of the endoscope internal components or reduction in diameter of the bending piece group 6a including the distal end piece 6f may be achieved.

It should naturally be understood that the present invention is not limited to the above-stated embodiment and various modifications or applications may be made without departing from the scope of the present invention. The above-described embodiment includes the invention in various stages, and various aspects of the invention may be extracted by appropriately combining a plurality of disclosed component elements. For example, in the case where the problem to be solved by the invention can be solved and advantages of the invention can be demonstrated even if some component elements are removed from all the component elements shown in the aforementioned one embodiment, the configuration without the removed component elements may be extracted as the invention. Furthermore, component members in different embodiments may properly be combined. The present invention is limited by attached claims but is not restricted by specific embodiments.

What is claimed is:

1. An endoscope comprising:
a cylindrical member disposed at a distal end of a bending portion provided on a distal end side of an insertion portion;
a wire inserted into the cylindrical member along a longitudinal axis of the insertion portion;
a protruding portion provided in the cylindrical member, the protruding portion being provided by cutting and bending a predetermined part of an outer peripheral surface of the cylindrical member in a direction of a central axis;
an outer peripheral surface-side concave provided on the protruding portion so as to allow disposition of a distal end portion of the wire; and
a guide groove provided in the protruding portion, the guide groove including an end part face provided at a predetermined position of the outer peripheral surface-side concave, the guide groove being elongated along the central axis and larger in width than a diameter of the wire, the guide groove being configured to guide the wire inserted into the cylindrical member to the outer peripheral surface-side concave.

2. The endoscope according to claim 1, wherein
a distance from the outer peripheral surface-side concave of the protruding portion to the outer peripheral surface of the cylindrical member is larger than the diameter of the wire.

3. The endoscope according to claim 1, wherein
a distance from an inner peripheral surface end provided on a proximal end surface of an outer peripheral-side opening of the cylindrical member to a proximal end of the outer peripheral surface-side concave on the end part face in the guide groove provided on the outer peripheral surface-side concave is larger than the diameter of the wire.

4. The endoscope according to claim 1, wherein
an amount of protrusion of the protruding portion from an inner peripheral surface of the cylindrical member is equal to the diameter of the wire.

5. The endoscope according to claim 1, wherein
the protruding portion includes a thin wall portion provided by firmly installing the wire at a predetermined position and then plastically deforming a central axis-side convex that is opposite to the outer peripheral surface-side concave in a direction of the outer peripheral surface of the cylindrical member.

6. The endoscope according to claim 1, wherein
the distal end portion of the wire is fixed to the outer peripheral surface-side concave by soldering.
